(19) United States
(12) Patent Application Publication
(10) Pub. No.: US 2002/0064555 A1
(43) Pub. Date: May 30, 2002

(54) Proton pump inhibitor formulation

(76) Inventors: Dan Cullen, Arvada, CO (US); Christopher L. Pelloni, Hatfield, PA (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/962,785

(22) Filed: Sep. 25, 2001

Related U.S. Application Data
(60) Provisional application No. 60/236,993, filed on Sep. 29, 2000.

(51) Int. Cl. A61K 9/56; A61K 31/4439

(52) U.S. Cl. 424/459; 514/338

(57) ABSTRACT

Pharmaceutical capsule dosage forms of benzimidazole proton pump inhibitors are prepared by enclosing one or several enteric coated compressed cores in a capsule shell. The inventive formulations are stable and have higher bioavailability of the active ingredient relative to pellet and granule containing formulations.

This application claims the benefit of Provisional Application No. 60/236,993 filed Sep. 29, 2000

### BACKGROUND

Benzimidazole compounds of the formula (I)

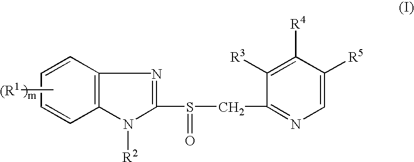

wherein R¹ to R⁵ are defined later herein, are known as gastric proton pump inhibitors and have utility in the treatment of gastric and duodenal ulcers, gastroesophageal reflux disease and other conditions associated with excess gastric acid secretion. Several of these compounds are commercially available and/or have been tested clinically, for example, omeprazole, lansoprazole, leminoprazole, pariprazole, rabeprazole and pantoprazole.

Although these compounds are reported to have a high degree of therapeutic utility, they are also reported to be highly acid labile. This has presented a problem to formulators of oral dosage forms, such as capsules, because the acid labile compounds react with both gastric acid in the stomach and with enteric coatings used to prevent the benzimidazole compound from coming into contact with gastric acid.

U.S. Pat. No. 4,786,505 reports solving this problem by formulating the benzimidazole compound (omeprazole) and an alkaline-reacting compound into pellets and coating the pellets with an inert subcoating and then an enteric coating. The alkaline reacting compound presumably increases stability by maintaining the benzimidazole compound in an alkaline environment and the inert subcoating prevents contact between the benzimidazole compound and the enteric coating.

U.S. Pat. No. 5,626,875 reports a stable formulation which does not contain an alkaline-reacting compound but which also utilizes an inert subcoat to prevent contact between the benzimidazole compound and the enteric coating. The formulation is prepared by coating spherical inert cores with a first layer of the benzimidazole compound, a non-alkaline water soluble polymer and non-alkaline excipients, followed by a second layer of the non-alkaline water soluble polymer and non-alkaline excipients, followed by a third layer which is an enteric coating.

According to the present invention neither the alkaline reacting compound nor the subcoat are required if the enteric coating is applied to a compressed core containing the active ingredient. Generally, such compressed cores are distinguished from known pellet formulations by being significantly harder and denser and by having a significantly lower surface area to volume ratio due to the signicantly reduced surface area for the same volume occupied. From one to six, preferably one to four, of such enteric-coated compressed cores are encapsulated in a capsule shell to provide a capsule dosage form which meets all of the stability and purity requirements necessary to be commercially marketed as a pharmaceutical product.

Suprisingly, the inventive approach to formulating benzimidazole compounds provides a stable formulation which has improved bioavailability relative to the commercially-available enteric coated pellet or granule containing formulations, such as the omeprazole product marketed as PRILOSEC or LOSEC capsules, the lansoprazole product marketed as PREVACID or the rabeprazole product marketed as ACIPHEX.

### DETAILED DESCRIPTION

The present disclosure describes inventive capsule dosage forms for benzimidazole proton pump inhibitors. The inventive capsule dosage forms provide improved bioavailability compared with known pellet- or granule-based dosage forms as well as appropriate stability for a commercial pharmaceutical dosage form.

The inventive capsule dosage forms are delayed-release pharmaceutical capsule dosage forms which comprise one or several enteric-coated, compressed cores encapsulated by a capsule shell, wherein the enteric-coated compressed core consists essentially of a mixture of a pharmaceutically acceptable carrier and an pharmceutically effective amount of a pharmaceutically active compound of the formula (I)

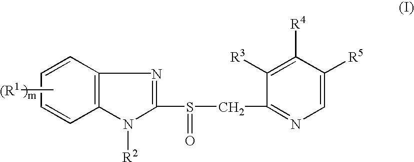

wherein R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulfinyl, R² is hydrogen, alkyl, acyl, carboalkoxy, carbamoyl, alkylcarbamoyl, dialkylcarbamoyl, alkylcarbonylmethyl, alkoxycarbonylmethyl or alkylsulfonyl, R³ and R⁵ are the same or different and each is hydrogen, alkyl, alkoxy or alkoxyalkoxy, R⁴ is hydrogen, alkyl, alkoxy which may optionally be fluorinated, or alkoxyalkoxy, and m is an integer of 0 through 4, or a pharmaceutically acceptable salt thereof; which mixture has been compressed at a pressure in the range from 350 to 1500 pounds into a compressed core and the compressed core is directly coated with an effective release-delaying amount of an enteric coating. Preferably, the compression pressure is in the range from 500 to 1200 pounds.

Generally, the capsule dosage form will contain 1 or several compressed cores. As a practical matter the upper limit is about 6 compressed cores per capsule. Although the capsule dosage form can contain from 1 up to about 6 compressed cores, it is preferable for the capsule dosage form to contain from 1 to 4 compressed cores, for example 1, 2, 3 or 4 compressed cores.

It is possible for the carrier to be essentially neutral meaning that it is not required for the carrier to function to keep an alkaline microenvironment within the compressed core. However, the carrier should not create an acidic microenvironment due to the acid lability of the benzimidazole compounds.

Essentially neutral carriers include fillers, surfactants, disintegrants, lubricants, binders and the like. Suitable fillers include lactose, sucrose, mannitol, dextrose, dextrates, sorbitol, dibasic calcium phosphate, microcrystalline cellulose, cellulose powder, starch, pregelatinized starch and the like. Suitable surfactants include polysorbates, sodium lauryl sulfate and polaxomers. Suitable disinegrants include crospovidone, sodium starch glycolate and croscarmellose sodium. Suitable lubricants include magnesium stearate, sodium stearyl fumarate and hydrogenated vegetable oil. Suitable binders include povidone, starch, dextrin and the like.

Generally, each compressed core has a volume in the range from about 13 to 1230 mm³ and a surface area to volume ratio of from 0.5 to 2.7 mm⁻¹,preferably 0.5 to 2.5 mm⁻¹, for example a volume in the range from about 25 mm³ to 450 mm³ or about 75 mm³ to 450 mm³ and a surface area in the range from about 50 mm² to 350 mm² or about 100 mm² to 350 mm² with a surface area to volume ratio of from about 0.5 to 2.5 mm⁻¹.

Generally, each compressed core will contain the same portion of the pharmaceutically active ingredient. Thus, if there are 4 compressed cores, each will contain 25% of the total dose, and, if there are 2 compressed cores, each will contain 50% of the total dose of active ingredient. However, variations are possible within the scope of the invention.

Normally, the benzimidazole compounds are provided in dosage forms containing from 10 to 50 mg of the active ingredient and each compressed core normally contains from 3 to 25 milligrams, for example 5 to 15 mg, of the pharmaceutically active compound. For example, omeprazole is marketed in 10, 20, 30 and 40 mg strengths and the 20 mg strength can comprise 4×5 mg compressed cores or 2×10 mg compressed cores and so on. As another example, lansoprazole is marketed in 15 and 30 mg strengths and the 30 mg strength can comprise 2×15 mg, 4×7.5 mg, 3×10 mg or 6×5 mg compressed cores and the 15 mg strength can comprise 3×5 mg, 2×7.5 mg or 1×15 mg compressed cores.

Preferably, the compound of formula (I) is selected from the group consisting of omeprazole, lansoprazole, leminoprazole, pariprazole, rabeprazole and pantoprazole; especially omeprazole or lansoprazole.

Enteric coatings suitable for application directly to the compressed core are well-known in the pharmaceutical arts. Generally, the enteric coating is a gastric resistant polymer such as cellulose acetate phthalate, hydroxypropylmethylcellulose acetate succinate, hydroxypropylmethylcellulose phthalate, polyvinylacetate phthalate, carboxymethylethylcellulose, acrylic acid polymers and copolymers, methacrylic acid polymers and copolymers. Copolymers of methacrylic acid and methacrylic acid methyl ester are especially useful as the enteric coating.

Although some discoloration may occur if an inert subcoating is absent, the subcoating is not necessary for stability purposes. Thus, capsule dosage forms wherein the enteric coating is applied directly (i.e. in the absence of a subcoating) to the compressed core are within the scope of the present invention.

The enteric coating is generally applied at a level which is effective to render the compressed core impermeable to gastric fluid.

There are four essential steps to preparing the inventive capsule dosage forms: mixing, compression, enteric coating and encapsulation.

The mixing step is carried out by known methods, preferably dry blending or wet granulation methods. Generally, the benzimidazole compound is dry blended with the carrier in a high or low sheer mixing apparatus, such as a vertical mixer, horizontal mixer, twinshell blender, double cone blender or a reciprocal blender, followed by de-agglomeration, roller compacted and milled to obtain a desirable particle size distribution. Alternatively, the mixing step is a wet granulation followed by drying, deagglomeration and milling. The milled material may be further blended with excipients, such as lubricants, to improve various properties.

The milled material is then compressed on a conventional tablet press, for example, a rotary tablet press, to yield a compressed core which is not friable and which has a hardness of about 3 Strong-Cobb units or greater.

The compressed core is then enteric coated by applying an effective amount of an enteric coating to render the tablet impermeable to gastric media. The coating operation is carried out in conventional or perforated coating pans, or may be carried out in a fluid bed apparatus.

The enteric coated compressed core is then filled into a capsule shell utilizing conventional encapsulation equipment with a tablet filling station. Such equipment is known in the art. A filler may be added to the capsule to eliminate rattling of the capsules in the capsule shell. If desired, the filler may contain additional pharmaceutical active ingredients to prepare a capsule dosage form containing a delayed-release proton pump inhibitor and an immediate release additional pharmaceutical agent.

The inventive formulations have improved bioavailability relative to pellet and granule formulations. Thus, the present invention especially relates to an omeprazole dosage form which has improved bioavailability relative to the omeprazole formulation which is the subject of U.S. Food and Drug Administration approved New Drug Application 19810, and to a lansoprazole dosage form which has improved bioavailability relative to the lansoprazole formulation which is the subject of U.S. Food and Drug Administration approved New Drug Application 20406. It is believed, although not certain, and without being bound to any particular theory, that the inventive formulations have improved bioavailability relative to the commercial formulations which contain enteric coated granules or pellets because a portion of the granules or pellets in the commercial products release their contents in the stomach and the active ingredient is decomposed before it is absorbed into the bloodstream.

The present invention further relates to a method of inhibiting the secretion of gastric acid in a mammalian subject which comprises administering to the subject a delayed-release, pharmaceutical capsule dosage form, which comprises one or more enteric-coated, compressed cores encapsulated by a capsule shell, wherein the enteric-coated compressed core consists essentially of a core which is a mixture of a pharmaceutically acceptable carrier and an effective amount of a pharmaceutically active compound of the formula (I)

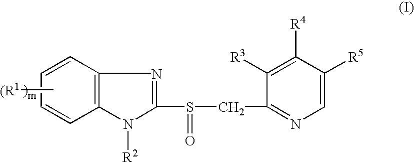

wherein R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulfinyl, R² is hydrogen, alkyl, acyl, carboalkoxy, carbamoyl, alkylcarbamoyl, dialkylcarbamoyl, alkylcarbonylmethyl, alkoxycarbonylmethyl or alkylsulfonyl, R³ and R⁵ are the same or different and each is hydrogen, alkyl, alkoxy or alkoxyalkoxy, R⁴ is hydrogen, alkyl, alkoxy which may optionally be fluorinated, or alkoxyalkoxy, and m is an integer of 0 through 4;

which mixture has been subjected to compression at a pressure in the range from 350 to 1500 pounds. The invention especially relates to a method wherein the bioavailability of the benzimidazole compound is enhanced relative to a pellet- or granule-containing reference formulation.

The present invention further relates to a process for preparing a enteric-coated, capsule dosage form containing a pharmaceutical effective amount of a compound of formula (I)

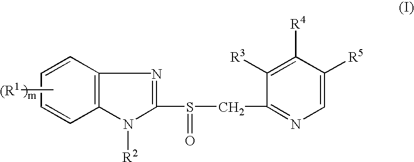

wherein R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulfinyl, R² is hydrogen, alkyl, acyl, carboalkoxy, carbamoyl, alkylcarbamoyl, dialkylcarbamoyl, alkylcarbonylmethyl, alkoxycarbonylmethyl or alkylsulfonyl, R³ and R⁵ are the same or different and each is hydrogen, alkyl, alkoxy or alkoxyalkoxy, R⁴ is hydrogen, alkyl, alkoxy which may optionally be fluorinated, or alkoxyalkoxy, and m is an integer of 0 through 4;

which consists essentially of the steps of

(i) compressing a mixture of a compound of formula (I) and a pharmaceutically acceptable carrier at a pressure in the range from 350 to 1500 pounds to form a compressed core which has a surface area to volume ratio of from 0.5 to 2.5 mm⁻¹;

(ii) coating the compressed core with an effective release-delaying amount of an enteric coating to form an enteric-coated compressed core; and

(iii) encapsulating from 1 to 4 enteric-coated compressed cores in a capsule shell to form a delayed-release capsule dosage form containing a pharmaceutically effective amount of a compound of formula (I).

### EXAMPLE 1

Omeprazole 20 mg capsules are prepared by the following procedure:

(1) The following ingredients are dry blended for 3 minutes in a 40 liter BOHLE blender:

| | |
|---|---|
| omeprazole | 1000 g |
| anhydrous lactose | 3695 g |
| microcrystalline cellulose | 600 g |
| sodium lauryl sulfate | 120 g |
| croscarmellose sodium | 224 g |

(2) After blending is complete, the blended composition is roller compacted in a FITZPATRICK IRL-520 CHILOSONATOR roller compactor at the following settings:

| | | |
|---|---|---|
| roll speed | 10 | rpm |
| roll pressure | 1400 | psi |
| mill speed | 2000 | rpm |
| mill screen | 79 | |
| vertical feed screw | 100 | rpm |
| horizontal feed screw | 20 | rpm |

(3) 60 grams of sodium stearyl fumarate and an equal portion of the compacted blend are passed through a 30 mesh screen.

(4) The blends from steps 2 and 3 are layered in a 40 liter BOEHLE blender and blended for 1.5 minutes on medium speed (setting 5).

(5) The resulting blend is compressed with a 11/64″ round, deep cup tooling at a target weight of 60 mg and a target hardness of 6 Strong-Cobb Units to yield compressed cores containing 10 mg of omeprazole.

(6) The compressed cores are coated with a mixture of 60% by weight EUGRAGIT L 30D 55 (suspension with 30% solids), 2% by weight polyethylene glycol (PEG 8000) and 38% purified water in a VECTOR COMPULAB coating pan at a spray pump setting of 8-10%.

The resulting enteric coated compressed cores have the following composition:

| | |
|---|---|
| CORE: | |
| omeprazole | 10.00 mg |
| anhydrous lactose | 36.95 mg |
| microcrystalline cellulose | 9.0 mg |
| sodium lauryl sulfate | 1.2 mg |
| croscarmellose sodium | 2.25 mg |
| ENTERIC COATING: | |
| EUGRAGIT L 30D 55 | 4.104 mg |
| polyethylene glycol | 0.213 mg |

(7) Two enteric coated compressed cores are placed into a capsule shell to yield a capsule dosage form containing 20 mg of omeprazole.

### EXAMPLE 2

This example describes the preparation of lansoprazole 15 and 30 mg capsules having the following composition:

| | |
|---|---|
| lansoprazole | 15 mg |
| lactose monohydrate | 29.8 mg |
| microcrystalline cellulose | 8.5 mg |
| polysorbate 80 | 0.6 mg |
| polyvinylpyrrolidone K-30 | 1.5 mg |
| croscarmellose sodium | 4 mg |
| sodium stearyl fumarate | 0.6 mg |

Preparation:

(1) Lansoprazole, lactose monohydrate, microcrystalline cellulose, polyvinylpyrrolidone and croscarmellose sodium are seived through a #40 sieve and mixed dry.

(2) The powder resulting from step (1) is granulated with water containing polysorbate 80 in a rapid mixer granulator.

(3) The granulate is passed through an 8 mm sieve, then dried at 40-45° C. for 2-3 hours and then passed through a #20 mesh seive.

(4) The granules resulting from step (3) are lubricated in a drum mixer—first with croscarmellose sodium and then with sodium stearyl fumarate.

(5) The lubricated granules are compressed using 4.3 mm round concave punches to a hardness of about 20N-35N at a average weight of 60 mg ±2% to yield compressed cores containing 15 mg of lansoprazole.

(6) The compressed core is coated with a formulation containing EUDRAGIT L30D 55, triethyl citrate and polyethylene glycol 400 in a ratio of 10:1:1 in a 15% aqueous suspension to yield enteric coated compressed cores wherein about 2.4 mg of EUDRAGIT L30D 55 is applied per compressed core.

(7) A color coat is applied to the enteric coated compressed core to yield a film coat which contains about 1.5 mg of hydroxypropylmethyl cellulose and 1.5 mg of titanium dioxide per core.

(8) One enteric coated compressed core is placed into a a size 3 capsule shell to yield a capsule dosage form containing 15 mg of lansoprazole, or two enteric compressed cores are placed into a size 1 capsule shell to yield a capsule dosage form containing 30 mg of lansoprazole.

We claim:

1. A delayed-release, pharmaceutical capsule dosage form, which comprises one or several enteric-coated, compressed cores encapsulated by a capsule shell, wherein the enteric coated compressed core consists essentially of a mixture of a pharmaceutically acceptable carrier and an pharmceutically effective amount of a pharmaceutically active compound of the formula (I) 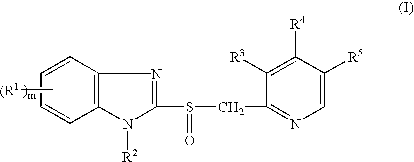
wherein R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulfinyl, R² is hydrogen, alkyl, acyl, carboalkoxy, carbamoyl, alkylcarbamoyl, dialkylcarbamoyl, alkylcarbonylmethyl, alkoxycarbonylmethyl or alkylsulfonyl, R³ and R⁵ are the same or different and each is hydrogen, alkyl, alkoxy or alkoxyalkoxy, R⁴ is hydrogen, alkyl, alkoxy which may optionally be fluorinated, or alkoxyalkoxy, and m is an integer of 0 through 4, or a pharmaceutically acceptable salt thereof; which mixture has been compressed at a pressure in the range from 350 to 1500 pounds to form a compressed core and the compressed core is directly coated with an effective release-delaying amount of an enteric coating.

2. A capsule dosage form of claim 1 wherein each compressed core has a surface area to volume ratio of from 0.5 to 2.5 mm⁻¹.

3. A capsule dosage form of claim 2 wherein each compressed core has a volume in the range from 13 to 1230 mm³.

4. A capsule dosage form of claim 2 wherein each compressed core has a volume in the range from about 25 mm³ to 450 mm³ and a surface area in the range from about 50 mm² to 350 mm².

5. A capsule dosage form of claim 2 which contains from 1 to 6 compressed cores.

6. A capsule dosage form of claim 3 which contains from 1 to 4 compressed cores.

7. A dosage form of claim 3 wherein the compound of formula (I) is selected from the group consisting of omeprazole, lansoprazole, leminoprazole, pariprazole, rabeprazole and pantoprazole.

8. A dosage form of claim 5 wherein the compound of formula (I) is selected from the group consisting of omeprazole, lansoprazole, leminoprazole, pariprazole, rabeprazole and pantoprazole.

9. A dosage form of claim 3 wherein the compound of formula (I) is omeprazole or lansoprazole.

10. A dosage form of claim 3 wherein the compound of formula (I) is omeprazole.

11. A dosage form of claim 7 where the enteric coating is a gastric resistant polymer selected from the group consisting of cellulose acetate phthalate, hydroxypropylmethylcellulose acetate succinate, hydroxypropylmethylcellulose phthalate, polyvinylacetate phthalate, carboxymethylethylcellulose, acrylic acid polymers and copolymers, methacrylic acid polymers and copolymers.

12. A dosage form of claim 11 wherein the enteric coating is a copolymer of methacrylic acid and methacrylic acid methyl ester.

13. A dosage form of claim 9 wherein all of the pharmaceutically active compound is contained in 1 or 2 compressed cores.

14. A dosage form of claim 10 wherein all of the pharmaceutically active compound is contained in 1 or 2 compressed cores.

15. A dosage form of claim 9 wherein the bioavailability of the benzimidazole compound is enhanced relative to a pellet- or granule-containing formulation.

16. A method of inhibiting the secretion of gastric acid in a mammalian subject which comprises administering to the subject a delayed-release, pharmaceutical capsule dosage form, which comprises one or more enteric-coated, compressed cores encapsulated by a capsule shell, wherein the enteric-coated compressed core consists essentially of a core which is a mixture of a pharmaceutically acceptable carrier and an effective amount of a pharmaceutically active compound of the formula (I) 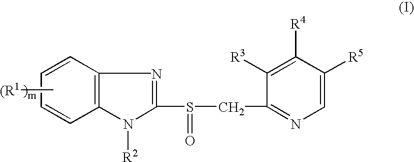
wherein R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulfinyl, R² is hydrogen, alkyl, acyl, carboalkoxy, carbamoyl, alkylcarbamoyl, dialkylcarbamoyl, alkylcarbonylmethyl, alkoxycarbonylmethyl or alkylsulfonyl, R³ and R⁵ are the same or different and each is hydrogen, alkyl, alkoxy or alkoxyalkoxy, R⁴ is hydrogen, alkyl, alkoxy which may optionally be fluorinated, or alkoxyalkoxy, and m is an integer of 0 through 4;
which mixture has been subjected to compression at a pressure in the range from 500 to 1200 pounds.

17. A method of claim 16 wherein the bioavailability of the benzimidazole compound is enhanced relative to a pellet- or granule-containing formulation.

18. A process for preparing a enteric-coated, capsule dosage form containing a pharmaceutical effective amount of a compound of formula (I) 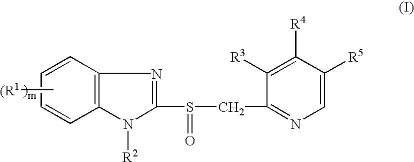
wherein R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulfinyl, R² is hydrogen, alkyl, acyl, carboalkoxy, carbamoyl, alkylcarbamoyl, dialkylcarbamoyl, alkylcarbonylmethyl, alkoxycarbonylmethyl or -alkylsulfonyl, R³ and R⁵ are the same or different and each is hydrogen, alkyl, alkoxy or alkoxyalkoxy, R⁴ is hydrogen, alkyl, alkoxy which may optionally be fluorinated, or alkoxyalkoxy, and m is an integer of 0 through 4;
which consists essentially of the steps of
(i) compressing a mixture of a compound of formula (I) and a pharmaceutically acceptable carrier at a pressure in the range from 350 to 1500 pounds to form a compressed core which has a surface area to volume ratio of from 0.5 to 2.5 mm⁻¹;
(ii) coating the compressed core with an effective release-delaying amount of an enteric coating to form an enteric-coated compressed core; and
(iii) encapsulating from 1 to 4 enteric-coated compressed cores in a capsule shell to form a delayed-release capsule dosage form containing a pharmaceutically effective amount of a compound of formula (1).

19. A process of claim 18 wherein the pressure is in the range from 500 to 1200 pounds.

20. A process of claim 19 wherein the compound of formula (I) is selected from the group consisting of omeprazole or lansoprazole.
